(19) United States
(12) Patent Application Publication
(10) Pub. No.: US 2019/0328648 A1
(43) Pub. Date: Oct. 31, 2019

(54) ANHYDROUS COSMETIC COMPOSITIONS COMPRISING LIPIDS, INORGANIC PARTICULATES, AND AN ALIPHATIC TACKIFIER

(71) Applicant: L'OREAL, PARIS (FR)

(72) Inventors: Cristina DUBCEAC, Linden, NJ (US); Lisa Ann VOORHEES - NORDHAUS, Middlesex, NJ (US); Roselin ROSARIO-MELENDEZ, New York, NY (US); Sam Bartlett MORRISON, Clark, NJ (US); Susan Ashley DESTENO, Springfield, NJ (US)

(73) Assignee: L'OREAL, PARIS (FR)

(21) Appl. No.: 15/963,591

(22) Filed: Apr. 26, 2018

(51) Int. Cl. A61K 8/81; A61K 8/37; A61K 8/26; A61K 8/25; A61Q 1/02

(52) U.S. Cl. CPC A61K 8/8111; A61K 8/37; A61K 8/26; A61K 8/0241; A61Q 1/02; A61K 2800/43; A61K 2800/31; A61K 8/25

(57) ABSTRACT

Compositions including an unsaturated aliphatic tackifier having a weight average molecular weight of less than about 10,000 daltons; an organic carbonate ester; a high melting point hydroxylated fatty acid ester; a swellable clay; boron nitride; a filler pigment and a color pigment are provided. The compositions have a combined concentration of lipids and inorganic particulates that is at least about 50% by weight and are substantially free of water. Methods of using the compositions are also provided.

### FIELD OF THE INVENTION

The present invention relates to cosmetic compositions comprising lipids and inorganic particulates.

### DISCUSSION OF THE BACKGROUND

Pigmented cosmetic compositions designed to conceal skin imperfections are known. Such compositions may take various forms, such liquids, creams, loose powders, pressed powders, hot pour compositions, sticks, etc. However, these compositions have one or multiple drawbacks in various properties.

The inventors of the instant invention have recognized that many of these prior art compositions do not simultaneously possess a combination of a properties that would be very advantageous for “spot treatment” of skin imperfections. For example, the present inventors have recognized that it would be desirable to have a composition that provides good tack and skin adhesion yet also has a pleasant gliding sensation when applied. The present inventors have separately and further recognized that it would be desirable for such a composition to also have shine and convenient application to the skin.

Accordingly, certain aspects of the present invention relate to a makeup and/or treatment composition for keratinous materials which can conceal skin imperfections and have surprisingly good cosmetic properties such as, for example, good tack and skin adhesion, a pleasant sensation upon application, an appropriate level of shine, and/or convenient application to the skin.

### SUMMARY OF THE INVENTION

The present invention relates to compositions that include an unsaturated aliphatic tackifier having a weight average molecular weight of less than about 10,000 daltons; an organic carbonate ester; a high melting point hydroxylated fatty acid ester; a swellable clay; boron nitride; a filler pigment; and a color pigment. The compositions have a combined concentration of lipids and inorganic particulates that is at least about 50% by weight and are substantially free of water.

According to another aspect of the invention, a method of making up a keratinous substrate includes a method of concealing imperfections on a keratinous substrate comprising topically applying the above-mentioned compositions to selected imperfections on the keratinous substrate.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

As used herein, all ranges provided are meant to include every specific range within, and combination of sub ranges between, the given ranges. Furthermore, notably the range description of the type “from about 1%, 2% or 3% to about 5%, 10% or 15%,” includes 1-5, 1-10, 1-15, 2-5, 2-10, 2-15, 3-5, 3-10, and/or 3-15. Other than in the operating examples, or where otherwise indicated, all numbers expressing quantities of ingredients and/or reaction conditions are to be understood as being modified in all instances by the term “about,” meaning within 10% to 15% of the indicated number.

“Actives basis” as used herein means considering only the particular component of ingredient (e.g., in a composition) and ignoring other chemically unrelated components that may be also be present in the same raw material source of that particular component.

“Film former” or “film forming agent” as used herein means any material such as, for example, a polymer or a resin that leaves a film on the substrate to which it is applied.

“Polymer” as used herein means a compound which is made up of at least two monomers.

“Keratinous materials” includes materials containing keratin such as hair, skin, eyebrows, lips and nails.

“Solids basis” as used herein means considering only components (e.g., in a composition) that are solid at room temperature and ignoring portions of the composition that are liquid, e.g., water and other volatile solvents.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 100° C.

“Non-volatile”, as used herein, means having a flash point of greater than about 100° C.

“Anhydrous” means the compositions contain less than 1% water. Preferably, the compositions of the present invention contain less than 0.5% water, and preferably no water.

All percentages of ingredients herein are listed on an actives basis unless specifically stated otherwise.

The composition of the present invention may be of various forms including liquid, paste, semi-solid, soft solid, solid, or self-supporting (e.g., molded/stick).

The composition of the invention generally include lipids, inorganic particulates, and an aliphatic tackifier and are substantially free of water. The lipids may (co)exist, for example in a fatty phase, such as one having inorganic particulates dispersed therein. The aliphatic tackifier may be dissolved or dispersed in the fatty phase. Many other optional ingredients such as other particulates, polymers and the like may also be dissolved or dispersed in the fatty phase.

Although the relative proportions of these components may vary, according to certain embodiments of the invention, the composition includes a concentration of lipids and inorganic particulates such that the total (combined) concentrations of lipids and inorganic particulates is at least about 50% by weight of the total composition. In certain embodiments the amount of lipids and inorganic particulates are present in a concentration by weight of at least about 60% by weight of the total composition, such as at least about 70%, such as at least about 80% by weight of the total composition.

According to certain other embodiments, the lipids and inorganic particulates are present in a weight ratio of lipid to inorganic particulate that is from about 1:2 to about 2:1.

According to certain other embodiments, the composition includes from about 15% by weight to about 60% by weight of lipids and from about from about 15% by weight to about 60% by weight of inorganic particulates. According to certain other embodiments, the composition includes from about 25% by weight to about 55% by weight of lipids and from about from about 25% by weight to about 55% by weight of inorganic particulates. According to certain other embodiments, the composition includes from about 35% by weight to about 55% by weight of lipids and from about from about 35% by weight to about 55% by weight of inorganic particulates.

The compositions and methods of the present invention can comprise, consist of, or consist essentially of the essential elements and limitations of the invention described herein, as well as any additional or optional ingredients, components, or limitations described herein or otherwise useful in personal care.

Unsaturated Aliphatic Tackifier

In accordance with the present invention, compositions include at least one unsaturated aliphatic tackifier having a molecular weight of less than about 10,000 daltons, such as less than about 5000 daltons, such as from about 1500 to about 4000. The inventors have found that the aliphatic tackifier provides enhanced skin adhesion suitable for use in spot treatment (so called “concealer” formulas). By “tackifier,” it is meant a material that enhances surface adhesion and may have a glass transition temperature less than about 0° C. (such as measured by DTA or DSC). By “aliphatic” is meant a hydrocarbon based compounds or material that are straight, branched, or cyclic, but are free of aromatic ring structures. According to certain notable embodiments, the aliphatic tackifier is unsaturated, such as a polybutene. Polybutene tackifiers may be made by polymerization of C4 olefins (e.g, primarily isobutene) and may be, for example, free-flowing, sticky with a honey-like consistency, or very tacky, semi-solid materials. Such synthetic hydrocarbon tackifiers may have a viscosity between about 1000 centistokes (cSt) and 5000 cSt when measured at 100° C. One notable aliphatic tackifier is INDOPOL H 1500, commercially available from Ineos Capital of London, England.

According to certain embodiments, the concentration of the unsaturated aliphatic tackifier in the composition is at least about 10% by weight, such as from about 10% to about 20%, such as from about 10% to about 15% by weight in the composition.

Boron Nitride

Compositions of the present invention include boron nitride. The inventors have found that boron nitride surprisingly allows the composition to take advantage of the adhesion and tack of the unsaturated aliphatic tackifier yet still provide a soft gliding sensation and a soft focus for the user. According to certain embodiments the boron nitride has an average particle size from about 5-15 microns.

One notable boron nitride suitable for use in the composition is SOFTOUCH BORON NITRIDE POWDER CC6058, commercially available from Momentive Performance Materials, Waterford, N.Y.

The concentration of the boron nitride in the composition may range from about 2% to about 10% by weight as from about 2% to about 5% by weight in the composition.

Swellable Clay

Compositions of the present invention include a swellable clay. By “swellable clay” it is meant a clay material that is capable of swelling in water. An example of a swellable clay are smectite clays. The crystal structure of the smectite group, is an octahedral alumina sheet between two tetrahedral silica sheets. In one notable embodiment, the swellable clay is bentonite. Bentonite is a rock formed of highly colloidal and plastic clays composed mainly of montmorillonite, a clay mineral of the smectite group, and is produced by in situ devitrification of volcanic ash. In addition to montmorillonite, bentonite may contain feldspar, cristobalite, and crystalline quartz. Bentonite has an ability to form thixotrophic gels with water, an ability to absorb large quantities of water. Variations in interstitial water and exchangeable cations in the interlayer space affect the properties of bentonite and thus the commercial uses of the different types of bentonite.

One notable swellable clay suitable for use in the composition is BENTONE GEL GTCC V, commercially available from Elementis Specialties, East Windsor, N.J. BENTONE GEL GTCC V is a dispersion of organically modified hectorite in caprylic/capric triglycerides with added propylene carbonate.

The concentration of the swellable clay in the composition may range from about 0.1% to about 2% by weight as from about 0.2% to about 0.65% by weight, such as from about 0.2% to about 0.45% in the composition.

Organic Carbonate Ester

Compositions of the present invention include an organic carbonate ester. The organic carbonate ester is believed to enhance the oil absorption ability of the swellable clay, thereby promoting good skin feel and product stability. According to certain embodiments of the invention, the organic carbonate ester is a C1-C4 alkylene carbonate such as ethylene carbonate or propylene carbonate and most notably, propylene carbonate.

The organic carbonate ester may be, as noted above added via the same raw material as the swellable clay. Alternatively, it may be added separately.

The concentration of organic carbonate ester in the composition may range from about 0.05% to about 0.5% by weight as from about 0.1% to about 0.3% by weight in the composition.

According to certain embodiments, the swellable clay and organic carbonate ester are present in a ratio by weight of swellable clay to organic carbonate ester that is less than about 3. According to certain other emobodiments, the ratio by weight of swellable clay to organic carbonate ester is less than about 2, such as from about 1 to about 1.75.

High Melting Point Hydroxylated Fatty Acid Ester

Compositions of the present invention include a high melting point hydroxylated fatty acid ester. By “high melting point hydroxylated fatty acid ester” it is meant a hydroxylated fatty acid ester having a melting point of at least 40° C. In certain embodiments the hydroxylated fatty acid ester has a melting point of at least about 50° C., such as at least about 60° C., such as at least about 80° C. The inventors have found that the high melting point hydroxylated fatty acid ester when used with the organic carbonate ester surprisingly allows the composition to resist oil syneresis and other adverse stability issues.

According to certain embodiments the high melting point hydroxylated fatty acid ester is a triester of a fatty acid and glycerol (fatty acid glycerol triester). According to certain embodiments the high melting point hydroxylated fatty acid ester has at least one C12 or greater carbon chain. In certain other embodiments, it has at least two hydroxyl groups. In certain other embodiments, the high melting point hydroxylated fatty acid ester is trihydroxystearin.

One notable hydroxylated fatty acid ester suitable for use in the composition is the hydroxystearin THIXCIN R, commercially available from Elementis Specialties, East Windsor, N.J.

The concentration of the hydroxylated fatty acid ester in the composition may range from about 0.1% to about 5% by weight, such as from about 0.25% to about 2%, such as from about 0.25% to about 1% by weight in the composition.

Inorganic Pigments

Compositions of the present invention include one or more inorganic pigments. As one skilled in the art will readily recognize,

“Inorganic particulate” means any finely divided material that is predominantly inorganic (including inorganic particulates having an organic or silicon-based coating, but excluding silicone elastomer particulates). Inorganic particulates are meant to encompass boron nitride (described above) as well as “filler pigments,” and “color pigments,” described below.

Filler Pigments

Compositions of the present invention include one or more filler pigments. The filler pigment provides one or more benefits including opacity/hiding powder to aid in concealing skin imperfections, modification of the texture of the formula, oil absorption, as well as possible mattifying and optical/soft focus effects. Suitable filler pigments include any of various inorganic pigments and the like that provide opacity and generally appear white or whitish on the skin and do not otherwise provide strong visible color. Examples of filler pigments include inorganic pigments such as titanium dioxide, talc, mica, silica, silica silylates, perlite, kaolin, as well as bismuth oxychloride, zinc oxide, among others. The filler pigments may be coated or unocoated. In certain notable embodiments the filer pigments in the composition include titanium dioxide, mica, silica silylate, and perlite. According to certain notable embodiments, the composition includes a hydrophobic silica such as silica silylate (particulate hydrophobic silica having surface trialkylsiloxyl groups).

Any of various cosmetic grades of filler pigments are suitable for use in compositions of the present invention. In certain embodiments the filler pigments have an average particle size in a range from about 1 micron to about 100 microns, such as from about 1 micron to about 10 microns.

The concentration of the filler pigments in the composition may range from about 10%, 15%, or 20% to about 25%, 30%, 40%, or 50% by weight, including all ranges and subranges therebetween.

Color Pigment

Compositions of the present invention include a color pigment. The color pigment provides hiding powder to aid in concealing skin imperfections while imparting some additional visible color. Suitable color pigments include any of various inorganic pigments such as iron oxides, ultramarine blue pigments, manganese violet, ferric ferrocyanide and chromium green pigments, and the like. The color pigments may be coated or unocoated.

Any of various cosmetic grades of color pigments are suitable for use in compositions of the present invention. In certain embodiments the color pigments have an average particle size in a range from about 1 micron to about 100 microns, such as from about 1 micron to about 10 microns.

The concentration of the color pigments in the composition may range from about 0.01%, 0.05%, or 0.5% to about 1%, 2%, 5%, or 10% by weight, including all ranges and subranges therebetween.

Lipids

As described above, compositions of the present invention include a high melting point hydroxylated fatty acid ester. In certain notable other embodiments, the composition includes other lipids. By “lipid” it is meant any of various fatty substances insoluble in water and including, for example, hydrocarbon based fatty substances or silicone-based fatty substances. These may be liquid or solid at room temperature and may be volatile or non-volatile.

Examples of suitable fatty substances include oil(s) and/or wax(es). As used herein, by “oils,” it is meant compounds having a melting point of less than about 30° C. and generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties.

Suitable examples of compounds of oils include vegetable oils (glyceryl esters of fatty acids, monoglycerides, diglycerides, triglycerides) and fatty esters. Specific non-limiting examples include, without limitation, esters such as isopropyl palmitate, isopropyl myristate, isononyl isonanoate C12-C15 alkyl benzoates, caprylic/capric triglycerides, ethylhexyl hydroxystearate, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others.

Suitable oils include volatile and/or non-volatile oils. Such oils can be any acceptable oil including but not limited to silicone oils and/or hydrocarbon oils.

According to certain embodiments, the compositions of the present invention may include one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C.

The concentration of oils in the composition may be from about 5%, 10% or 15% by weight to about 20%, 30% or 50% by weight, including all combinations of such ranges, relative to the total weight of the composition.

Compositions of the present invention may include wax. As used herein, “wax” is intended to mean a lipophilic fatty compound that is solid at room temperature (about 25° C.) and atmospheric pressure (760 mm Hg, i.e., 105 Pa), which undergoes a reversible solid/liquid change of state and which has a melting point of greater than 30° C., and in some embodiments, greater than about 55° C. up to about 120° C. or even as high as about 200° C.

The wax may change from the solid to the liquid state reversibly, and/or may have a hardness of more than 0.5 MPa at ambient temperature, and an anisotropic crystalline organization in the solid state.

The term wax may include waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fiber waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes (linear, low molecular weight polyethylene waxes), waxes obtained by Fischer-Tropsch synthesis, waxy copolymers and their esters, and silicone and fluoro waxes.

The term wax may further include high melting point hydrogenated oils of animal or plant origin. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils.

While in certain embodiments, compositions of the present invention include from about 1% to about 10% wax. However, in certain other embodiments, compositions of the present invention are have only limited amounts of wax, such as less than about 5% by weight, such as from about 1% to about 4% by weight.

In certain embodiments of the invention, the composition includes both wax and hydrophobic silica. In certain other embodiments the wax and hydrophobic silica are present in a weight ratio of total wax to total hydrophobic silica that is greater than about 4, such as greater than about 4.5, such as from about 4.5 to about 10, such as from about 4.5 to about 6.5. In certain other embodiments, the composition not only has such a total wax to total hydrophobic silica, but also the swellable clay and organic carbonate ester are present in a ratio by weight of swellable clay to organic carbonate ester that is less than about 3.

In certain embodiments of the invention, the composition may include other lipids which may not fall into the definition of wax or oils, such as fatty materials that are semi-solid, pastes or paste-like at room temperature. Suitable examples include shea butter, hydrogenated butters, and any of various partial esters of diglycerin with medium chain fatty acids, such as Bis-Diglyceryl Polyacyladipate-2, commercially available as SOFTISAN 649 from Cremer Oleo of Hamburg, Germany.

The total concentration of lipids in the composition may be from about 10%, 15%, 20%, 25%, or 35% by weight to about 40%, 55% or 60% by weight, including all combinations of such ranges, relative to the total weight of the composition.

Other Ingredients

Compositions of the present invention may optionally include other functional ingredients such as those that can be readily dissolved, dispersed or suspended in the composition. These may include other particulate materials (organic, silicone-based); polymers such as for thickening/rheology modifying; preservatives; dyes, fragrances; antioxidants; sunscreens; vitamins; and the like. A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

While in certain embodiments, compositions of the present invention include a surfactant or emulsifier, in certain other notable embodiments, the composition is substantially free of surfactants and emulsifiers.

The other ingredients may be present in the composition in concentrations up to about 20%, such as from about 0%, 2%, or 5% to about 10%, 15%, or 20%, including all ranges and subranges therebetween.

According to preferred embodiments of the present invention, methods of treating, caring for and/or making up a keratinous material by applying compositions of the present invention to the keratinous material in an amount sufficient to treat, care for and/or make up the keratinous material are provided. According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to cover or hide defects associated with keratinous material, or to enhance the appearance of keratinous material. The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1—Inventive Composition

Compositions consistent with the ingredients and concentrations below were prepared:

| | | INVENTIVE | INVENTIVE |
|---|---|---|---|
| FUNCTION/ | | EXAMPLE 1 | EXAMPLE 2 |
| TYPE | INGREDIENT | CONCENTRATION BY WT. | |
| Swellable clay + | Stearlkonium | 3% | 0.5% |
| organic carbonate | hectorite, | (BENTONE | Disterdimonium |
| ester + | caprylic/capric | GEL GTCCV) | hectorite + |
| triglycerides | triglyceride, | | 0.16% |
| | Propylene | | propylene |
| | carbonate | | carbonate |
| High melting | Trihydroxystearin | 0.5% | 0.5% |
| point | | | |
| hydroxylated | | | |
| fatty acid ester | | | |
| Inorganic | Boron nitride and | 40% | 40% |
| Particulate | other inorganic | | |
| | particulates | | |
| Oils and other | Fatty acid | 40% | 43% |
| lipids | triglycerides, | | |
| | other fatty | | |
| | acid esters, | | |
| Waxes | Beeswax and | 3.2% | 2% |
| | other waxes | | |
| Unsaturated | polybutene | 14% | 14% |
| aliphatic | | | |
| tackifier | | | |

For inventive example 1, the oils and waxes were melted to create a uniform liquid mixture. After allowing to cool to approximately 60° C., the swellable clay including propylene carbonate (BENTONE GEL GTCC) and trihydroxystearin were mixed in with high shear until homogeneous. The remaining solid ingredients (filler pigments and color pigments and unsaturated aliphatic tackifier) were then added to the container with stirring at approximately 60° C. until a homogeneous fluid composition was obtained. The composition was then allowed to cool to room temperature. The consistency was such that compositions could be filled into tubes that could be squeezed to release the composition. The compositions were applied to skin and had a surprisingly pleasant feel upon application, good tack, appropriate shine, and high skin coverage.

For Inventive Example 2, a similar process was followed, except that both the clay and propylene carbonate were added as individual raw materials before the addition of the solid ingredients. Furthermore, the ratio of by weight of swellable clay to organic carbonate ester was 3.1, as compared to Inventive Example 1, where the ratio of by weight of swellable clay to organic carbonate ester was 1.5. The resulting composition had lower shine than Inventive Example 1.

### Example 2—Comparative Composition

A comparative composition with the ingredients and concentrations below was also prepared

| | | COMPARATIVE |
|---|---|---|
| | | EXAMPLE 1 |
| | | CONCENTRATION |
| FUNCTION/TYPE | INGREDIENT | BY WT. |
| Swellable clay + | Stearlkonium | 0.5% |
| organic carbonate | hectorite, | Disterdimonium |
| ester + | caprylic/capric | hectorite + |
| triglycerides | triglyceride, | 0.16% propylene |
| | Propylene | carbonate |
| | carbonate | |
| High melting point | Trihydroxystearin | 0% |
| hydroxylated fatty | | |
| acid ester | | |
| Inorganic | Boron nitride and | 40% |
| Particulate | other inorganic | |
| | particulates | |
| Oils and other | Fatty acid | 43% |
| lipids | triglycerides, | |
| | other fatty | |
| | acid esters, | |
| Waxes | Beeswax and | 2.4% |
| | other waxes | |
| Unsaturated | polybutene | 14% |
| aliphatic | | |
| tackifier | | |

For Comparative Example 1, a similar process to Inventive Example 2 was followed, except that trihydroxysterin was omitted. The resulting composition had poor phase stability and low coverage.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. accordingly, the description is not intended to limit the invention to the form disclosed herein. Also, it is intended to the appended claims to construed to include alternative embodiments.

What is claimed is:

1. A composition, comprising:
an unsaturated aliphatic tackifier having a weight average molecular weight of less than about 10,000 daltons;
an organic carbonate ester;
a high melting point hydroxylated fatty acid ester;
a swellable clay;
boron nitride;
a filler pigment; and
a color pigment, wherein the composition has a combined concentration of lipids and inorganic particulates that is at least about 50% by weight and wherein the composition is substantially free of water.

2. The composition of claim 1 wherein the unsaturated aliphatic tackifier is polybutene.

3. The composition of claim 1 wherein the unsaturated aliphatic tackifier is present in a concentration of at least about 10% by weight.

4. The composition of claim 1 wherein the organic carbonate ester is a C1-C4 alkylene carbonate.

5. The composition of claim 1 wherein the high melting point hydroxylated fatty acid ester has a melting point above 50° C.

6. The composition of claim 1, wherein the high melting point hydroxylated fatty acid ester is a fatty acid glycerol triester.

7. The composition of claim 1, wherein the high melting point hydroxylated fatty acid ester is trihydroxystearin.

8. The composition of claim 1, wherein the composition comprises from about 15% by weight to about 60% by weight of lipids and wherein the composition comprises from about from about 15% by weight to about 60% by weight of inorganic particulates.

9. The composition of claim 1, wherein the composition comprises from about 25% by weight to about 55% by weight of lipids and wherein the composition comprises from about from about 25% by weight to about 55% by weight of inorganic particulates.

10. The composition of claim 1, wherein the lipids and inorganic particulates are present in a weight ratio of lipid to inorganic particulate that is from about 1:2 to about 2:1.

11. The composition of claim 1, wherein the composition has a concentration of wax that is less than about 5% by weight.

12. The composition of claim 1, wherein the composition further comprises hydrophobic silica.

13. The composition of claim 12, wherein the composition further comprises wax and wherein the wax and hydrophobic silica are present in a weight ratio of wax to hydrophobic silica that is greater than about 4.

14. The composition of claim 1, wherein the swellable clay and organic carbonate ester are present in a ratio by weight of swellable clay to organic carbonate ester that is less than about 3.

15. The composition of claim 13, wherein the swellable clay and organic carbonate ester are present in a ratio by weight of swellable clay to organic carbonate ester that is less than about 3.

16. A method of concealing imperfections on a keratinous substrate comprising topically applying the composition of claim 1 to selected imperfections on the keratinous substrate.
